(19) United States
(12) Patent Application Publication
(10) Pub. No.: US 2010/0167259 A1
(43) Pub. Date: Jul. 1, 2010

(54) APPARATUS AND METHOD FOR THE PRESERVATION OF PANCREATIC TISSUE AND ISLET CELLS FOR TRANSPLANTATION

(75) Inventors: Shinichi Matsumoto, Arlington, TX (US); Tetsuya Ikemoto, Richardson, TX (US)

Correspondence Address: CHALKER FLORES, LLP, 2711 LBJ FRWY, Suite 1036, DALLAS, TX, 75234, US

(73) Assignee: BAYLOR RESEARCH INSTITUTE, Dallas, TX (US)

(21) Appl. No.: 12/645,886

(22) Filed: Dec. 23, 2009

Related U.S. Application Data
(60) Provisional application No. 61/223,595, filed on Jul. 7, 2009.
(60) Provisional application No. 61/140,908, filed on Dec. 26, 2008.

(51) Int. Cl. A01N 1/02; C12M 1/00

(52) U.S. Cl. 435/1.1; 435/284.1; 435/303.1

(57) ABSTRACT

The present invention includes compositions and methods for the preparation, preservation and storage of organs (e.g., pancreatic islet cells) for transplantation by storing organs in which the organs are suspended in a solution for maintaining viability and the organ or cells are cooled in a refrigeration unit for the entire duration of storage in which the average temperature in the apparatus does not vary by more than 2 degrees centigrade from the set temperature.

### CROSS-REFERENCE TO RELATED APPLICATIONS

This application is related to U.S. Provisional Application Ser. No. 61/140,908, filed Dec. 26, 2008, the entire contents of which are incorporated herein by reference.

### TECHNICAL FIELD OF THE INVENTION

The present invention relates in general to the field of pancreatic islet transplantation, and more particularly, to a new apparatus and methods for improving the preservation of pancreatic tissue and islet cells prior to transplantation.

### STATEMENT OF FEDERALLY FUNDED RESEARCH

None.

### BACKGROUND OF THE INVENTION

Without limiting the scope of the invention, its background is described in connection with islet cell transplantation.

One method for improving islet preservation is taught in U.S. Pat. No. 5,679,565, issued to Mullen, et al. Mullen teaches a method, a solution and a chamber for the preparation and storage of pancreatic islets. The method includes contacting a pancreas with a warm collagenase solution, digesting the pancreas in the warm collagenase solution to form a warm digest, adding cold preservative solution to the warm digest, agitating the warm digest/cold preservative solution at a temperature between about 0 and 15 degrees C., to thereby further digest the partially digested pancreas included in the warm digest, to form a cold digest and collecting liquid from the cold digest to form isolated islets. The cold preservative solution and a pancreatic islet preservative solution of the present invention include D-mannitol, K-lactobionate and a buffer.

Another method is taught in United States Patent Application No. 20070009880, filed by Toledo, et al., for methods and solutions for storing donor organs. Briefly, Toldeo, et al., teach methods of preserving, storing and transplanting mammalian donor organs. The method includes the cooling of refrigeration preservation, loading pre-freezer preservation, cryopreservation, and washing solutions at least containing polyvinylpyrrolidone, a calcium channel blocker, a nucleoside, potassium chloride, polyethylene glycol, at least one amino acid, and a steroid to a temperature of 2° to 4° C. and/or of 0° to 2° C., harvesting a donor organ, perfusing it with one or more of the solution, immersing it in one or more of the solutions and storing it at a temperature above 0° C. or at a temperatures below 0° C., −20° C., −80° C. and −196° C. The cryopreservation solution also contains cryopreservative agents. Preserved organs may be transplanted directly from refrigeration storage or from freezer storage by cooling the washing refrigeration preservation solutions to 2° to 4° C., perfusing the organ with washing solution and then preservation solution, and transplanting it.

Yet another method and composition is taught in United States Patent Application No.

20020164795, filed by Gen, for a storage agent for preservation of an animal cell, tissue or organ, and preserved process of the same. Briefly, application relates to the storage agent for preservation of an animal cell or organ and preserved process. Ordinary method of cell storage is employed preserving method by freezing at extra low temperature of −196° C. and the survival ratios of cells after thawing and fusion is low, about 10 to 30%. The period of validity is a very short time of 12 to 72 hours. The storage agent can make protein stabilize to protein type storage agent, and prevent, treat and improve organ injury caused on an organ transplant operation by adding the polyphenol.

### SUMMARY OF THE INVENTION

Despite the compositions and agents used to improve preservation of organs for transplant, a need remains to improve the length of storage and the quality of the organs following their extraction, processing and transportation. As the availability of organs for transplantation has not kept up with the demand for organs, improved methods are necessary to maximize the limited pool of available donor organs.

The present invention increases not only the time of storage available for viable organs but also increases the quality of the organs for transplantation. Increasing the storage time, viability and organ quality is essential to allow for testing of the organs, time of transport and success rate for transplantation.

In one embodiment, the present invention includes an apparatus and method for storing organs or tissues in which the organs or tissues are suspended in a solution for maintaining viability and the organ, tissues or cells are cooled in a refrigeration unit for the entire duration of storage in which the average temperature in the apparatus does not vary by more than 2 degrees from the set temperature. In one aspect, the apparatus cools the organ or the tissue from body temperature to about 4° C. within 18 minutes. In another aspect, the apparatus further comprises one or more portals for a preservation gas selected from CO₂, N₂ or O₂. In another aspect, the apparatus comprises one or more probes that determine the organ or cell temperature. In another aspect, the apparatus vary the temperature in the apparatus no more that 1 degree from the set temperature. In another aspect, the apparatus has a set temperature of greater than 0, 1, 2, 3, 4, 5 or 6 degrees centigrade. In one aspect, the organ or the tissue comprises at least a portion of a liver, a lung, a cornea, a muscle, a heart, a pancreas, pancreatic islets, a kidney, a breast, an eye, an ear, a bone or a bone marrow. In another aspect, the organ or the tissue is treated during storage with one or more active agents that will enhance organ transplant. In another aspect, the organ or the tissue is treated during storage with one or more active agents selected from antibodies, enzymes, steroids, antibiotics, proteases, nucleases, vectors, nucleic acids, proteins, peptides, lipids, carbohydrates, salts, minerals, vitamins, buffers, gases, electrical impulses, mechanical stress (extension and/or compression), radiation or toxins. In yet another aspect the viability of the stored organ or the tissue is at least 80%. In a certain aspect the viability of the stored organ or the tissue is 100%, 95%, 90%, 85%, 80%, 75%, 70%, 60%, 50%, 40% and 30%.

In another embodiment, the present invention includes a method of preserving an organ or a tissue by obtaining the organ or the tissue for transplant, placing the organ or the tissue in a preservation solution, cooling the organ or the tissue to a pre-selected temperature and maintaining the organ or the tissue at the pre-selected temperature for the duration of storage at a temperate that does not vary more that 2 degrees centigrade from the pre-selected temperatures. In one aspect, the apparatus cools the organ or the tissue from body temperature to about 4° C. within 18 minutes. In another aspect, the apparatus further comprises one or more portals for a preservation gas selected from CO₂, N₂ or O₂. In another aspect, the apparatus comprises one or more probes that determine the organ, the tissue or cell temperature. In another aspect, the apparatus varies the temperature in the apparatus no more that 1 degree from the set temperature. In another aspect, the apparatus has a set temperature of greater than 0, 1, 2, 3, 4, 5 or 6 degrees centigrade. In one aspect, the organ or the tissue comprises at least a portion of a liver, a lung, a cornea, a muscle, a heart, a pancreas, pancreatic islets, a kidney, a breast, an eye, an ear, a bone or a bone marrow. In another aspect, the organ or the tissue is treated during storage with one or more active agents that will enhance organ transplant. In another aspect, the organ is treated during storage with one or more active agents selected from antibodies, enzymes, steroids, antibiotics, proteases, nucleases, vectors, nucleic acids, proteins, peptides, lipids, carbohydrates, salts, minerals, vitamins, buffers, gases, electrical impulses, mechanical stress (extension and/or compression), radiation or toxins. In yet another aspect the viability of the stored organ or the tissue is at least 80%. In a certain aspect the viability of the stored organ or the tissue is 100%, 95%, 90%, 85%, 80%, 75%, 70%, 60%, 50%, 40% and 30%.

More particularly, the present invention can also be used in conjunction with improved compositions and methods of preparing a transplantable islet preparation, such as, harvesting the pancreas or the pancreatic tissue from a donor; injecting one or more pancreatic ducts with ET-Kyoto solution or equivalent thereto; isolating pancreatic β-islet cells; and treating the patient with a human interleukin-1 antagonist at the time of islet transplant. In one embodiment, wherein the pancreatic β-islet cells are treated with a suitable collagenase, e.g., a human collagenase. In one specific example, the islets are processed in ET-Kyoto solution after their extraction from the pancreas. In one aspect, human interleukin-1 antagonist is selected from: one or more modifiers of interleukin-1 beta (IL-1β) gene transcription; one or more modifiers of IL-1β gene translation; one or more siRNAs that target the expression of IL-1β; one or more IL-1β receptors blockers; one or more interleukin-1 receptor antagonist proteins; one or more interleukin-1 receptor antagonist peptides; one or more active agents that modify the release of IL-1β; one or more antibodies that neutralize IL-1β; one or more antibodies that blocks an IL-1β receptor; one or more recombinant, naturally occurring IL-1β receptor antagonists; one or more anion transport inhibitors, lipoxins and alpha-tocopherol that inhibit the release of IL-1β; one or more opioids that inhibits a proteolytic enzyme that converts the inactive IL-1β precursor to its mature, active form; one or more antibodies that neutralizes the biological function of IL-1β, mixtures and combinations thereof. In one specific example, the IL-1β antagonist is anakinra The method may further include concurrently providing the patient with a Tumor Necrosis Factor antagonist, selected from inhibitors of gene transcription, inactivated Tumor Necrosis Factors, Tumor Necrosis Factor Receptor blockers and soluble Tumor Necrosis Factor Receptor.

In one aspect the isolated pancreatic β-islet cells have a recovery rate of at least 35%. In another aspect the recovery rate of the isolated pancreatic β-islet cells is 100%, 95%, 90%, 85%, 80%, 75%, 70%, 60%, 50%, 40%, 35%, 30%, 25%, and 20%. In yet another aspect the isolated pancreatic β-islet cells have a purity of at least 70%. In one aspect the purity of the isolated pancreatic β-islet cells is 100%, 95%, 90%, 85%, 80%, 75%, 70%, 60%, 50%, 40%, 35%, 30%. In a specific aspect the isolated pancreatic β-islet cells have a viability of at least 80%. In certain aspects the viability of the isolated pancreatic β-islet cells is 100%, 95%, 90%, 85%, 80%, 75%, 70%, 60%, 50%, 40% and 30%.

Another aspect of the present invention is a method of preparing a transplantable islet preparation, the method including the steps of: harvesting the pancreas or the pancreatic tissue from a donor; injecting one or more pancreatic ducts with ET-Kyoto solution or equivalent thereto; isolating pancreatic β-islet cells from the harvested pancreas or the pancreatic tissue in the presence of a trypsin inhibitor; and treating the patient with a human interleukin-1 antagonist at the time of islet transplant. Examples of trypsin inhibitors include serum α-1 antitrypsin, a lima bean trypsin inhibitor, a Kunitz inhibitor, a ovomucoid inhibitor or a soybean inhibitor.

Yet another embodiment of the present invention is a method of preparing a transplantable islet preparation, by harvesting the pancreas or pancreatic tissue from a donor; isolating pancreatic β-islet cells isolating pancreatic β-islet cells from the harvested pancreas or the pancreatic tissue in the presence of a trypsin inhibitor; and treating the patient with a human interleukin-1 antagonist and a Tumor Necrosis Factor antagonist at the time of islet transplant. In one aspect the transplantable islet preparation has a viability of at least 80%.

### BRIEF DESCRIPTION OF THE DRAWINGS

For a more complete understanding of the features and advantages of the present invention, reference is now made to the detailed description of the invention along with the accompanying figures and in which:

FIG. 1 compares pig pancreas preservation at 37° C. of the prior art for 48 hours, a pig pancreas stored at 4° C. using conventional refrigeration and a pig pancreas stored using the preservation apparatus of the present invention also at 4° C. for 48 hours;

FIG. 2 is a graph that shows the number of islets per field comparing the prior art, the pancreas stored using preservation solution at 37° C., 4° C. and the present invention (islet cell number per field (40×);

FIG. 3 compares the morphology of human islet cells kept for 48 and 72 hours at 37° C., 22° C., 4° C. and 4° C. using the apparatus and method of the present invention;

FIG. 4 compares the viability of human islet cells kept for 48 hours at 37° C., 22° C., 4° C. or 4° C. using the apparatus and method of the present invention by staining with two different dyes;

FIG. 5 shows the temperature changes during the measurements. All temperatures were within 0.5° C. errors during measurements. The temperature achieved setting point within 5 minutes for 22.0° C., 37.0° C. and 4.0° C. and maintained stable. The temperature of KFC was decreased rapidly to 6.0° C. and gradually got down to 1.0° C. at the rate of 0.3° C./hour as the primary settings. All temperatures were measured by thermometer 54 II with 80PK-1 K-type Bead thermocouple;

FIG. 6 shows the morphology of preserved porcine islets for 24, 48 and 72 hours. Large islets tended to vanish in group 1 (at 37.0° C. preservation) and 2 (at 22.0° C. preservation) after 48 hours. Between low temperature settings, islets in group 3 (at 4.0° C. preservation) seemed to have more unclear borders than these in group 4 (Keep and Fresh cooling system, arrow). Original magnification 200×;

FIG. 7A is a plot showing the porcine islet recovery rate were calculated by dithizone staining The formula: Post preserved islet number/freshly isolated islet number (0 hours) (%). Low temperature settings (at 4.0° C. preservation and Keep and Fresh cooling system) could maintain high recovery rate. At 48 hours, islet recovery rates were 48.7±28.6% in group 1 (at 37.0° C. preservation), 46.6±15.5% in group 2 (at 22.0° C. preservation), 61.5±20.0% in group 3 (at 4.0° C. preservation) and 73.9±17.3% in group 4 (Keep and Fresh cooling system. P-value of KFC vs. 37.0° C. and 22.0° C. was <0.01, vs. 4.0° C. was <0.05, respectively). At 72 hours, islet recovery rates were 35.8±18.5% in group 1, 31.1±16.6% in group 2, 43.5±14.3% in group 3 and 61.0±22.0% in group 4, (P-value of KFC vs. 37.0° C. was <0.01, vs. 22.0° C. was <0.001, vs. 4.0° C. was <0.05 respectively, Newman-Keuls Test);

FIG. 7B is a graph showing the evaluation of porcine islet purity when islet equivalents were counted. At 24 hours, the purities dropped to 85.0±10.0% in group 1, 83.3±7.6% in group 2, 82.5±5.0% in group 3 and 85.0±9.4% in group 4. At 48 hours, the purities were 75.8±20.6% in group 1, 78.3±2.9% in group 2, 76.7±5.8% in group 3 and 84.5±9.9% in group 4, and at 72 hours, the purities were 68.6±23.8% in group 1, 73.3±14.7% in group 2, 77.5±8.7% in group 3 and 84.0±9.6% in group 4. There were significant differences in islet purity between Group 4 and 1 at 48 hours, at 72 hours (P-value was <0.01 at 48 hours, <0.05 at 72 hours. Newman-Keuls Test);

FIG. 7C shows the porcine islet cell viabilities at pre-purification (Pre), 24, 48 and 72 hours after the preservation were measured by Trypan blue staining At 24 hours, islet viabilities at 37.0° C., 22.0° C,4.0° C. and KFC decreased to 82.1±6.2% , 85.0±5.7%, 86.0±3.3% and 91.1±3.3%, respectively. At 48 hours, that of 37.0° C. ,22.0° C,4.0° C. and KFC decreased to 80.7±0.2%, 85.2±6.4%, 83.7±6.6% and 90.5±5.4%, respectively (P-value of KFC vs. 37.0° C. was <0.05. Dunnett's test. FIG. 7C). At 72 hours, that of 37.0° C., 22.0° C., 4.0° C. and KFC decreased to 78.0±9.1%, 80.0±4.6%, 82.0±1.8% and 89.1±2.6%, respectively (P-value of KFC vs. 37.0° C. was <0.05. Dunnett's test. FIG. 7C); and

FIG. 8 is a plot showing the stimulation index as a measure of the in vitro function of preserved porcine islets for 72 hours by KFC. Stimulation index (SI) were calculated, and compared with SI of the islets preserved at 37.0° C. (as a conventional preservation setting) for the same period. Mean SI of islets preserved at 37° C. for 72 hours was 1.4±0.4, by KFC was 3.0±2.1, was significantly higher preserved by KFC (P<0.03, unpaired t-test, three independent studies).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Diabetes mellitus (DM) type 1 is a disease with significant social and economic impact. The prevalence of the disease in the United States is about 120,000 in individuals aged 19 or less and 300,000 to 500,000 at all ages and 150 million worldwide. There are 30,000 new cases diagnosed each year in the United States. DM is one of the most frequent chronic diseases in children in the United States¹. The cost of treatment and complications of this disease in the United States is 90 billion dollars a year.

Islet cell transplantation (ICTx) is a promising therapy for type 1 diabetes mellitus (T1DM), however, the limitation of its widespread use is due to some critical issues². One of these issues is that ICTx requires large quantities of islets for achieving insulin free status³. Multi-donor-one-recipient transplantation is required because of this need for large quantities of islets to yield effective ICTx.

Another issue is a severe ICTx donor shortage in some countries, such as Japan⁴. It is sometimes difficult in these countries to find cadaveric donors both for clinical ICTx and for basic research. The inventors have previously reported a domestic shipping of clinical human islet⁵, however, there are many issues for international shipping of clinical human islets including FDA regulation and long term storage. In addition, once islet transplantation becomes the standard therapy, donor shortage will remain major issue due to the large number of type 1 diabetic patients. Currently there are more than one million type 1 diabetic patients, and the number of organ donations was less than 8,000 per year in the USA.

Islet transplantation using porcine islets is an attractive alternative to resolve the issue. Indeed, some reports have shown the possibility of using porcine islets for clinical ICTx^{(6,7)}. Porcine islets also have some difficulties. First, porcine islets are fragile^{(8,9)}. This fragility is due to the fact that porcine islet cells do not have a firm capsule compared to human islets⁽¹⁰⁾. Therefore isolated porcine islet cells are easily weakened or destroyed by enzymes such as trypsin and collagenase. These enzymes weaken the porcine islets to various stresses such as the shearing stress of the centrifuge, cell culture and hypoxic conditions. Effective preservation method for fragile porcine islets is necessary for their clinical application in ICTx.

There have been many reports of effective and innovative methods for low-temperature organ preservation^{(11,12)}. For pancreas, Obermaier et al. recently reported that pancreas preservation for the prevention of pancreatic ischemia/reperfusion injury is effective at 4° C. and at other temperatures as well¹³. However, the majority of published reports are for whole organs and limited types of cells there are relatively few reports for cell suspensions even though it seemed that low temperature is most effective for porcine islet preservation¹⁴⁻¹⁶. Moreover, the suitable temperature settings for islet preservation are still controversial¹⁷. Therefore, it is unclear which temperature setting is best for islet and how is the most stable preservation method for islets, especially porcine islets. Fujiya Co., (Tokushima, Japan) developed a novel cooling system that successfully kept freshly harvested plants and fruits fresh for over 180 days. Preserved plants and fruits were formed to be ‘hibernation status’. This cooling system is called “KEEP AND FRESH cooling system” (KFC). The cooling system can control the temperature of the material by stepwise cooling with minimum errors, ranging from −20.0° C. to room temperature using a computer with frequent sensing of the internal and surface temperatures of the materials and the internal temperature of refrigerator. The present invention demonstrates the application of the KFC system for effective preservation of porcine islets.

Islet isolation: Pancreata were procured from pigs at Owen Co., Ltd. as research settings from June 2008 to September 2008 in Texas, USA. After removal of the vessels, fat, connective tissues and a part of the connecting lobe, these pancreata were transported immediately to Baylor Institute for Immunology Research (BIIR) with two-layer method (oxygenated perfluorocarbon and University of Wisconsin solution), isolated and purified by Ricordi method and COBE 2991 cell processor previously described by the inventors in Matsumoto S, Noguchi H, Naziruddin B, et al., Improvement of pancreatic islet cell isolation for transplantation., Proc (Rayl Univ Med Cent) 2007;20(4):357-362.

Culture media: Islets were cultured in the culture media for islet. Briefly, this culture media contents CMRL culture media (Sigma-Aldrich, USA) with Human Serum Albumin, 1M Sodium Hydroxide and Sodium bicarbonate. These media were sterilized with filtration (2.4 μm, USA) and stored in the normal refrigerator at 4 C. Islets were preserved in 12 well tissue culture plate (Falcon, USA) with these media (put 2,000-3,000 IEQ islet/well) for the evaluation of islet recovery, purity and viability. Islets were also preserved in culture flasks for static incubation. No medium changes were undergone during the culture.

Islet preservation and temperature settings of the cooling system: Preservation settings for islet were divided to 4 groups. Group 1 is that islets were cultured in the 37° C., 5% CO2 incubator condition. Group 2 is that islets were cultured in the 22° C., 5% CO₂ incubator condition. Group 3 is that islets were preserved at 4° C. in a normal refrigerator. Group 4 is that islets were preserved in the settings of “Rapid cooling condition”; rapid cooling from room temperature (RT) to 4° C. and step wise cooling at 0.5° C./hour decreasing rate to 1.5° C.

Temperature measurements: Each temperature is measured by thermometer (54II, Fluke, USA) with 80PK-1 K-type Bead thermocouple. Long term temperature is analyzed by Fluke View Forms software.

Morphology: All islets were undergone histological examination with the fluorescence of diacetate and Hematoxylin-Eosin staining under microscope.

Islet Cell count and viability: Islet number and viability were counted as described in Matsumoto S, Noguchi H, Yonekawa Y, Okitsu T, Iwanaga Y, Liu X, et al., Pancreatic islet transplantation for treating diabetes. Expert Opin Biol Ther 2006;6(1):23-37, relevant portions incorporated herein by reference. Briefly, islet were counted under microscope with the fluorescence with Dithizone dye for at least two samples, after washing islet with culture media and taking 100 μL samples from 5 mL. Islet numbers were converted to islet equivalent (IE) as previously described. Cell viability was assessed with using membrane perm-selectivity dye FDA/PI under the fluorescence microscope with the staining At least two samples of islets were assessed for viability using 1% Trypan blue (Sigma Chemical Co.) for at least two samples.

Static incubation (in vitro function): In order to evaluate islet cell function after preservation by KFC in vitro, static incubation were performed as described previous¹⁸. Briefly, islet aliquots (50-100 IEQ) were incubated in parallel with either 2.8 or 20 mM glucose for 2 hours at 37° C. Insulin concentrations in supernatants were assessed by ELISA (Alpco, Salem, N.H.). DNA content of islet pellets was measured by fluorimetry for normalization of insulin concentrations. Glucose stimulated insulin release was expressed as stimulation index (SI). SI was calculated as the ratio of insulin released after exposure to high glucose over the insulin released in basal condition.

Statistics: Descriptive statistics were presented as mean±S.E., median with range for quantitative variables and number (percentages) for qualitative variables. Univariate analysis was performed by using one way analysis of variation (ANOVA), Kruskal-Wallis test, Welch test, Newman-Keuls test, Dunnett's test and unpaired t-test whenever appropriate. A p-value of <0.05 was considered statistically significant and all p-values reported were two-sided. All analyses were performed in State Mate III for Windows.

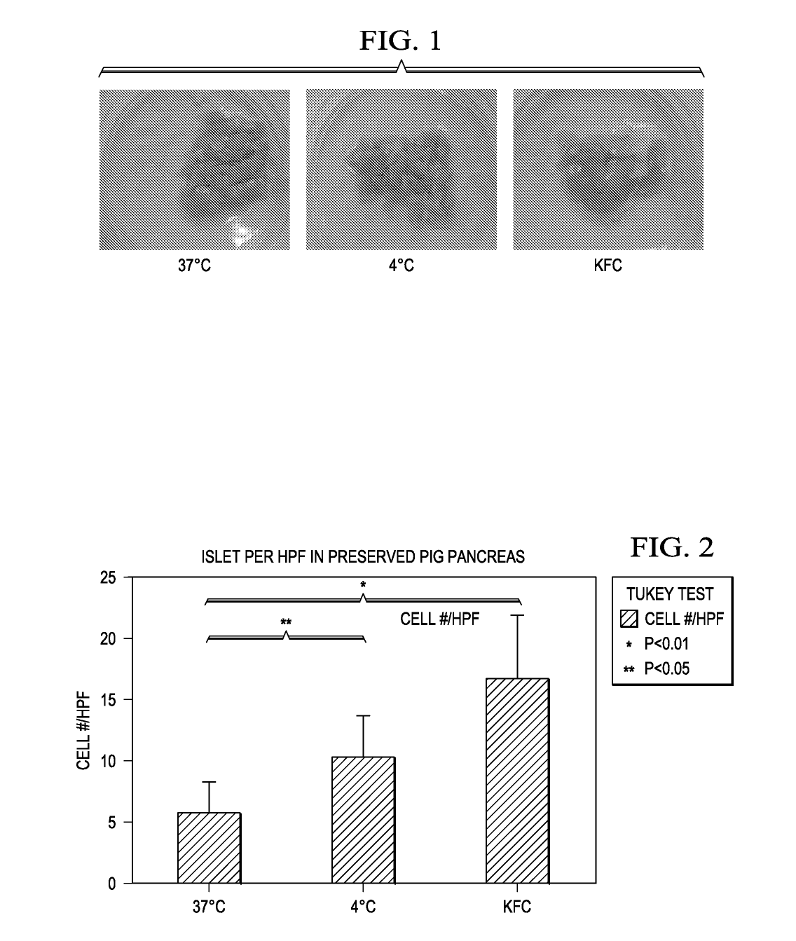

FIG. 1 compares pig pancreas preservation at 37° C. of the prior art for 48 hours, a pig pancreas stored using a conventional refrigeration unit at 4° C. and the preservation apparatus of the also at 4° C. for 48 hours. Briefly, eight (8) grams of islet cells were cut into three pieces and stored in UW solution at the listed temperature. The pancreas kept using the present invention most closely resembled a freshly isolated pig pancreas.

FIG. 2 is a graph that shows the number of viable islets per field comparing the prior art, the pancreas stored using preservation solution at 37° C., 4° C. and the present invention (islet cell number per field (40×). It was found that the number of islets were significantly higher in the group preserved using the present invention (labeled KFC) as compared to 37° C. or standard 4° C. refrigeration.

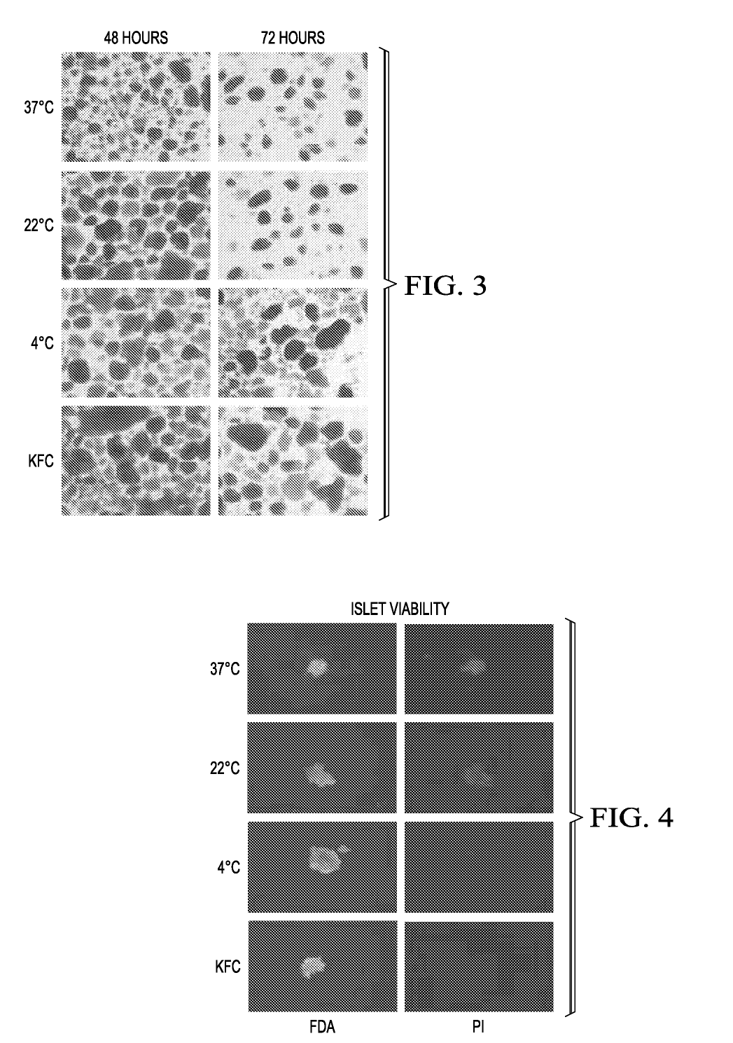

FIG. 3 compares the morphology of human islet cells kept for 48 and 72 hours at 37° C., 22° C., 4° C. or 4° C. using the apparatus and method of the present invention. It was found that the apparatus and method of the present invention was able to preserve human islets at the highest levels when compared to other storage methods and devices. Briefly, human islet cells were presented in culture medium at the listed temperatures, with or without, the present invention for 48 or 72 hours.

FIG. 4 compares the viability of human islet cells kept for 48 hours at 37° C., 22° C., 4° C. or 4° C. using the apparatus and method of the present invention by staining with two different dyes. Human islet cells were isolated at stored in media at the listed temperatures and for the listed times. Again, the apparatus and methods of the present invention show higher viability than current methods for preservation and storage.

The islet isolation data is shown in Table 1. Seven porcine pancreata were used for islet isolation. Mean pancreata weight after removal the connective lobe was 153.3±44.5 g. Mean warm ischemic time (defined as the interval between the time the pigs were killed and the time the pancreata were preserved in cold storage solution) was 45.6±7.9 min. Mean cold ischemic time (defined as the interval between the time the pancreata were preserved in cold storage solution and the time the pancreata were removed from preservation solution for isolation) was 121.3±2.2 min. Mean time of collagenase perfusion by an electric pump was 10.7±1.5 min. Phase I time (defined as the interval between the time of initiation of circulation of solution and the time of initiation of collection of tissue. (19)) was 10.4±2.6 min, and phase II time (defined as the interval between the end of Phase I and the end of islet collection. (19) was 40.7±5.7 min. Mean islet equivalent after purification (IE, high pure fraction only) was 603,805.2±291,752.0 IE. Mean purity and mean viability were 93.4±5.2% and 98.0±3.0% respectively.

TABLE 1
Porcine islet isolation date.

| | |
|---|---|
| Pancreas weight (g)* | 153.3 ± 44.5 |
| WIT (minutes) | 45.6 ± 7.9 |
| CIT (minutes) | 121.3 ± 2.2 |
| Perfusion time** | 10.7 ± 1.5 |
| Phase I digestion time*** (minutes) | 10.4 ± 2.6 |
| Phase II collection time****(minutes) | 40.7 ± 5.7 |
| Mean islets IE, pre purification | 1,049,654.6 ± 290,590.5 |
| Mean islets IE, post purification | |
| (Total) | 784,206.4 ± 254,520.5 |
| (High pure fraction^{$}) | 603,805.2 ± 291,752.0 |
| Mean islet IE/g, post purification^{$$} | 4904.2 ± 2352.7 |
| Mean purity^{#} (%) | 93.4 ± 5.2 |
| Mean viability^{#}(%) | 98.0 ± 3.0 |

Pig n = 7.
WIT: warm ischemic time, CIT: cold ischemic time (defined as from the time when procured pancreata were put into iced cooler boxes to the starting time of isolation).
IE: islet equivalent.
*Pancreas weight for processing, a part of connection lobe, fat and connecting tissues were removed,
**Perfusion: the time for Collagenase injection by electric pump,
***Phase I collection: the time between initiation of circulation of solution and initiation of collection of tissue,
****Phase II collection: the time between the end of Phase I and the end of islet collection. ^{$}High pure fraction: defined as ^{$$}islet IE/g: Purified islet IE/pancreas weight for processing, ^{#}Purity and viability from high pure fraction.

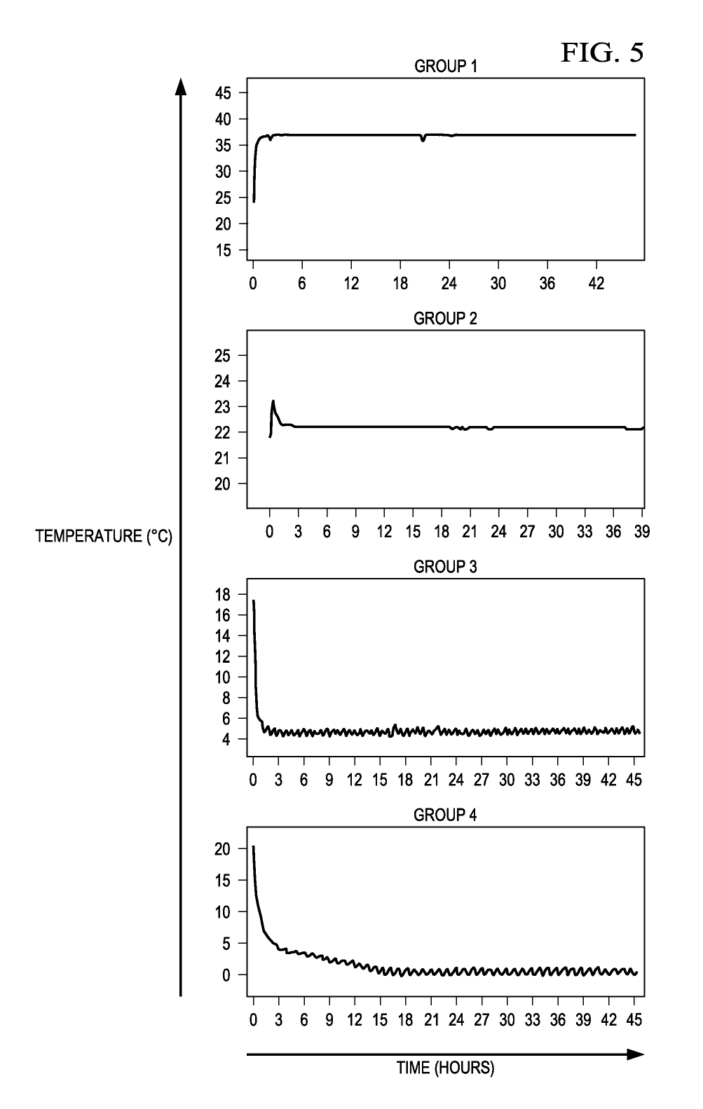

All temperatures were within 0.5° C. of the starting temperature during measurements (FIG. 5). The temperature achieved the set point within 5 minutes for 22.0° C., 37.0° C. and 4.0° C. and was stably maintained. The temperature of the KFC was decreased rapidly to 6.0° C. and gradually got down to 1.0° C. at the rate of 0.3° C./hour as the primary settings.

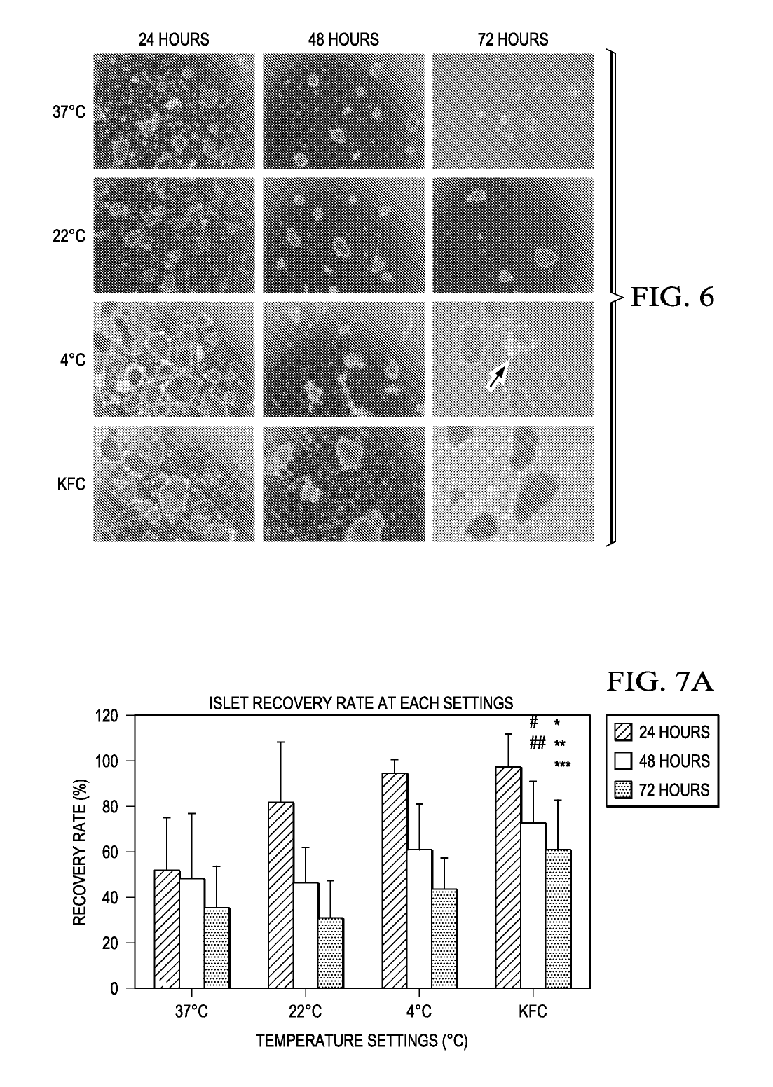

There were not apparent morphologic differences among each group of islets at 24 hours. However, gradually large islets disappeared in groups 1 (37° C.) and 2 (22° C.) after 48 hours. Between low temperature settings, islets in group 3 (4° C.) seemed to have more unclear borders than these in group 4 (FIG. 6, arrow).

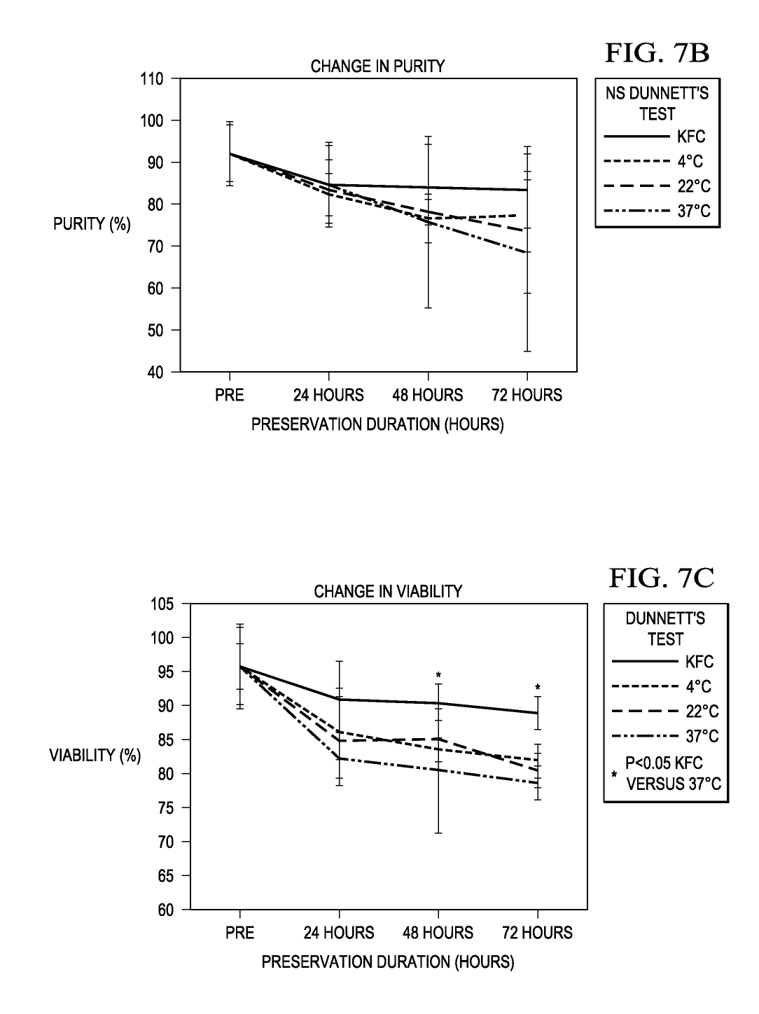

At 24 hours, islet recovery rates were 51.8±23.0% in group 1 and 82.5±26.0% in group 2 (FIG. 7A). On the other hand, recovery rate were much higher for low temperature cultures such as the use at 4° C. and KFC. These were 95.4±5.75% (4° C.) and 97.5±14.2% (KFC). Low temperatures could maintain high recovery rate. These tendencies were more significant after 48 hours. At 48 hours, islet recovery rates were 48.7±28.6% in group 1, 46.6±15.5% in group 2, 61.5±20.0% in group 3 and 73.9±17.3% in group 4. At 72 hours, islet recovery rates were 35.8±18.5% in group 1, 31.1±16.6% in group 2, 43.5±14.3% in group 3 and 61.0±22.0% in group 4, (P-value of KFC vs. 37.0° C. was <0.01, vs. 22.0° C. was <0.001, vs. 4.0° C. was <0.05 respectively. Newman-Keuls Test, FIG. 7A).

Islet purity was evaluated when islet equivalents were counted. At 24 hours, the purities dropped to 85.0±10.0% in group 1, 83.3±7.6% in group 2, 82.5±5.0% in group 3 and 85.0±9.4% in group 4. At 48 hours, the purities were 75.8±20.6% in group 1, 78.3±2.9% in group 2, 76.7±5.8% in group 3, and 84.5±9.9% in group 4, and at 72 hours, the purities were 68.6±23.8% in group 1, 73.3±14.7% in group 2, 77.5±8.7% in group 3 and 84.0±9.6% in group 4. There were significant differences in islet purity between Group 4 and 1 at 48 hours, at 72 hours (P-value was <0.01 at 48 hours, <0.05 at 72 hours. Newman-Keuls Test, FIG. 7B).

Cell viabilities at pre-purification (Pre), 24, 48 and 72 hours after the preservation were measured. Mean viability at pre-purification was 95.2±6.2%. At 24 hours, islet viabilities at 37.0° C., 22.0° C., 4.0° C. and KFC decreased to 82.1±6.2% , 85.0±5.7%, 86.0±3.3% and 91.1±3.3%, respectively. At 48 hours, viability decreased to 80.7±0.2% (37° C.), 85.2±6.4% (22° C.). 83.7±6.6% (4° C.) and 90.5±5 .4% (KFC), (P-value of KFC vs. 37.0° C. was <0.05 Dunnett's test, FIG. 7C). At 72 hours, the viability decreased to 78.0±9.1% (37° C.), 80.0±4.6% (22° C.), 82.0±1.8% (4° C.) and 89.1±2.6% (KFC), (P-value of KFC vs. 37.0° C. was <0.05. Dunnett's test, FIG. 7C).

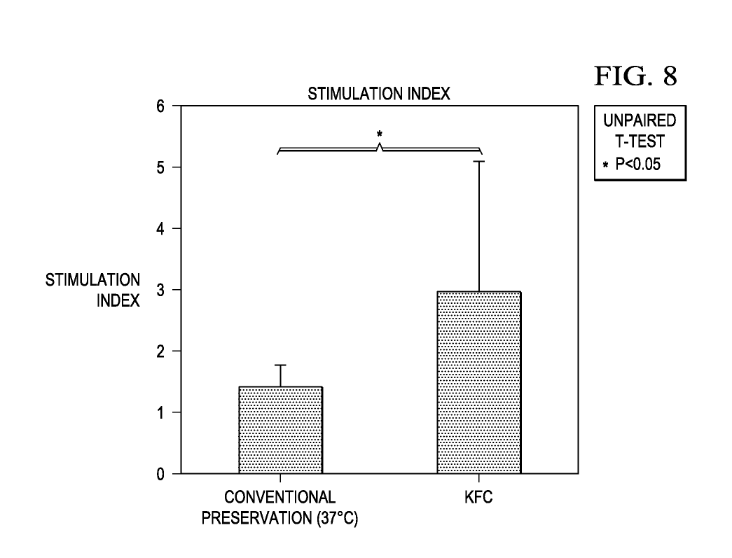

The inventors studies the in vitro function of porcine islets preserved for 72 hours by KFC. The stimulation index (SI) was calculated as described previously, and compared with SI of the islets preserved at 37.0° C. (as a conventional preservation setting) for the same period. Mean SI of islets preserved at 37° C. for 72 hours was 1.4±0.4, and by KFC was 3.0±2.1, which was significantly higher (P<0.03, unpaired t-test, FIG. 8).

The present invention can be used with novel preservation solutions, such as: (a) use of interleukin-1 blockade in the recipient of pancreatic islet cell transplants, (b) ductal preservation of the donor pancreas at the time of organ procurement by the preservative solution ET-Kyoto, and/or (c) the use of trypsin inhibition during donor pancreas digestion. ET-Kyoto solution, and the modifications thereto, include trehalose as a nonreducing disaccharide that stabilizes the cell membrane under various stressful conditions. Two variants on ET-Kyoto solution have different electrolyte contents, e.g., Na 100 mmol/L, K 44 mmol/L (so-called “extracellular” solution) and an “intracellular type” IT-Kyoto solution, e.g., Na 20 mmol/L, K 130 mmol/L, with trehalose at 35 gr/l. A non-limiting list of solutions that may be used with the present invention, are summarized in Table 2.

TABLE 2
List of preservation solutions.

| Solution | E-C | C-S | UW | LPD-G | ET-Kyoto | IT-Kyoto | nEt-Kyoto | C |
|---|---|---|---|---|---|---|---|---|
| Na+ | 10 | 17 | 30 | 165 | 100 | 20 | 107 | 100 |
| K+ | 115 | 115 | 125 | 4 | 44 | 130 | 44 | 15 |
| Mg++ | 5 | 5 | 5 | 2 | — | — | — | 13 |
| Ca++ | — | — | — | — | — | — | — | 0.25 |
| Cl— | 15 | 15 | — | 101 | — | — | — | — |
| CO3H— | 10 | 10 | — | — | — | — | — | — |
| PO4H2— | 58 | 58 | 25 | 36 | 26 | 25 | 25 | — |
| SO4═ | 5 | 5 | 5 | — | — | — | — | — |
| Glucose | 195 | — | — | 56 | — | — | — | — |
| Gluconate | — | — | — | — | 100 | 100 | 100 | — |
| Lactobionate | — | — | 100 | — | — | — | — | 80 |
| Adenosine | — | — | 5 | — | — | — | — | 1 |
| Glutamine | — | — | 3 | — | — | — | — | 1 |
| Alopurinol | — | — | 1 | — | — | — | — | 1 |
| Trehalose | — | — | — | — | 120 | — | 120 | — |
| Raffinose | — | — | 30 | — | — | — | — | — |
| Dextran 40(g/L) | — | — | — | 20 | — | — | — | — |
| Mannitol(g/L) | — | 37.5 | — | — | — | — | — | 60 |
| EDTA(g/L) | — | 0.075 | — | — | — | — | — | — |
| HES(g/L) | — | — | 50 | — | 30 | 30 | 30 | — |
| NAC | — | — | — | — | — | — | 10 | — |
| Db c-AMP | — | — | — | — | — | — | 2 | — |
| Nitroglycerine | — | — | — | — | — | — | 0.44 | — |
| pH | 7.4 | 7.4 | 7.4 | 7.4 | 7.4 | 7.4 | 7.4 | 7.3 |
| Osmolarity(**) | 355 | 420 | 325 | 335 | 370 | 370 | 600 | 360 |

E-C: Euro-Collins. C-S: Collins-Sacks. UW: University of Wisconsin - Beltzer. LPD-G: Low potassium Dextran - Glucose. ET-K: Extracellular-type Kyoto. IT-K: Intracellular-type Kyoto. nET-K: new ET-K; C: Celsior. EDTA: ethylenediaminetetraacetic acid. HES: Hydroxyethyl starch. NAC: N-acetylcysteine. Db c-AMP: Dibutyl cyclic AMP. All concentración in mMol/L, except (*)gr/L. (**)Osmolarity is expressed Osm/L.

Examples trypsin inhibitors include, but are not limited to, serum α-1 antitrypsin, a lima bean trypsin inhibitor, a Kunitz inhibitor, a ovomucoid inhibitor or a soybean inhibitor. To date there are no mechanical devices able to effectively adjust the dose of insulin injected according to the serum glucose levels in patients with DM. This leads to less-than-perfect sugar control, with episodes of hypoglycemia which can be dangerous.

Pancreas Transplantation—Benefits: Pancreas transplantation is a well-established treatment for type 1 DM. It is performed concomitantly with kidney transplantation [Simultaneous pancreas and kidney transplantation (SPK)], after kidney transplantation [“pancreas after kidney” (PAK)] or pancreas transplant alone (PTA). Simultaneous pancreas and kidney transplantation accounted for 75% of the pancreata transplanted in United States in 1999 and remains the procedure of choice for management for otherwise fit Type 1 diabetic patients under the age of 50 with renal failure¹⁹. The indications for PTA, which make up less than 10% of the total numbers, are less objective but include life-threatening hypoglycemia unawareness necessitating continual presence of a caregiver and aggressive diabetic neuropathy. Relief of hypoglycemia unawareness is the most convincing reason to accept the risks of lifetime immunosuppression. It is this same group of patients selected for PTA who are also considered appropriate candidates for isolated islet cell transplantation.

The major achievements with pancreatic transplantation are insulin-independency and the avoidance, halting or regression of some of the complications related to DM. Life-style benefits from successful pancreas transplantation are unquestioned, and long-term normoglycemia can be achieved²⁰⁻²². Perhaps the greatest benefit with respect to diabetic secondary complications is the improvement in autonomic and peripheral neuropathy; better cardiac function leads to better patient survival²³. Not only is nerve conduction velocity improved, indicating neuronal repair within nerve sheaths, but also conduction amplitude is improved, indicative of axonal regeneration²⁴ Transplantation must occur, however, before the onset of severe sensor motor neuropathy for the patient to derive the benefit. Usually, diabetic retinopathy does not improve post-transplant, as 90% of SPK patients already having permanent damage at time of transplantation²⁵.

Pancreas Transplantation—Morbidity and Mortality: Pancreas transplantation is a well-established surgical procedure. It is considered a major surgical procedure associated with morbidity and mortality. Additional morbidity and mortality is related to the inherent immunosuppression therapy. The technique used requires en bloc transplantation of the whole pancreatic organ with both the exocrine and endocrine component together with the duodenal loop.

The specific complications related to the surgical procedure are vascular, anastomotic²⁶. The most recent data suggests that technical failure rate is approximately 8% for SPK, 13% for PAK, and 11% for PTA. Graft thrombosis (typically venous) occurs in 2-14% of cases resulting in early graft loss²⁷.

Specific complications are related to the type of intestinal drainage of the allograft: enteric or to the urinary bladder. With bladder drainage; complications include immediate postoperative hematuria, urinary leaks, urinary reflux pancreatitis, metabolic acidosis and dehydration from the secretion of fluid and bicarbonate by the exocrine pancreas into the bladder, and sterile cystitis due to the effect of the exocrine pancreatic enzymes on the bladder and urethral epithelium. In 8% to 23% cases, these complications necessitate surgical conversion to enteric drainage²⁸. With enteric drainage, the major complication is an anastomotic intestinal leak with intra-abdominal abscess formation, potentially leading to sepsis, multi-organ failure and death. A large number of complications mentioned above are related to the exocrine part of the transplanted pancreas or the transplanted duodenal loop. Despite the intense immunosuppression commonly used, the rejection rate after pancreas transplantation is around 30%, with 10% graft loss. Graft survival nationwide, as recorded by UNOS, is 88.5% at 3 months, 80% at one year, 52.9% at 3 years and 40.7% at 5 years. Results are better with kidney-pancreas transplants (87.7%, 83.8%, 77.2% and 67.5%, respectively). During a ten-year period (1991-2000), the annual death rate range was 36.3 to 82.3 per 1000 patients for pancreas transplants and 31.1 to 63.2 per 1000 patients with kidney-pancreas transplants²⁹.

Pancreatic Islet Cell Transplantation—an Alternative to Whole Organ Pancreas Transplantation: The emerging alternative to whole organ pancreas transplantation is pancreatic islet cell transplantation (ICT). The process is based on the enzymatic isolation of the pancreatic islets of Langerhans from an organ procured from a cadaveric donor³⁰⁻³²; the islets obtained are injected into the liver of the recipient via percutaneous catheterization of the portal venous system³³. This procedure allows the selective transplantation of the insulin-producing cell population avoiding open surgery as well as the transplantation of the duodenum and the exocrine pancreas and their related morbidity.

There are currently two trends in islet cell transplantation, using the immediate and delayed infusion approach. The immediate transplantation focuses on the use of the shortest time possible between islet isolation and islet infusion. An alternative method implies short-term culture of the islets after the isolation and before transplantation. This ensures increased purity of the islet isolate while it does not affect the viability and the function of the islets and seems to yield good results while the procedure is performed in a semi-elective setting^{34,35}.

Different anatomic locations were tried for the engrafting of the islet cells³⁶⁻³⁸. Currently, the portal vein is the preferred site of infusion, given the relative ease of access, the high venous flow with a double circulation system (arterial and portal venous) of the liver. The liver has a good regenerative capacity and is one of the major sites of insulin action. The liver site also seems to confer some immunological privilege to the islets. When compared to the whole organ pancreas transplant, the ICT has reduced surgical risk, is quicker and less expensive, is performed as an outpatient procedure and has therefore gained good patient acceptance.

The initial efforts with ICT had only modest results. The immunosuppression regimen was similar to the one used in solid organ transplantation, based on high dose steroids and calcineurin inhibitors—both agents with diabetogenic effects³⁹. The results improved markedly with the changes in the manipulations of the islets^{30,32} and the change in immunosuppression, thus avoiding the higher doses of steroids and using sirolimus, tacrolimus and dacluzimab initiated by the investigators group at the University of Alberta in Edmonton, Canada. Their protocol requires, in general, two islet cell infusions to attain the critical cell mass necessary to achieve insulin-independency. The changes in treatment were adopted as the “Edmonton Protocol”, which is used in several transplant centers worldwide^{33,40}. A recent report from the Edmonton group showed that 65 patients have received islet transplant at this center and 44 patients became insulin independent³. At five year follow-up ˜80% showed presence of C-peptide indicating functioning transplanted islets, however, only ˜10% remained insulin free. Similar results have been reported from other centers within USA⁴¹. In another recent advancement in this field, the Minnesota group have shown that marginal dose of islet cells isolated from a single donor pancreas are sufficient to achieve insulin independence in severely affected type 1 diabetic patients⁴².

The morbidity related to the procedure includes complications related to the liver puncture, portal vein cannulation and elevation of the liver function tests (LFT). Complications related to the liver puncture are subcapsular or intra-parenchymal bleeding, intraperitoneal bleeding (cumulative frequency: 4% necessitating blood transfusion), gallbladder puncture (2%), biliary leaks (1%). Pneumothorax and/or hemothorax are exceedingly rare. Formation of fatty patches in the liver (steatosis) has been reported⁴³. It is likely that the incidence of these complications may be lowered with the use of smaller catheters and the use of ultrasonographic guidance to access the portal vein²⁴ and fibrin glue for closing hole of puncture in the liver. Complications of the portal vein cannulation and infusion include portal vein branch thrombosis (2%) and partial minor portal vein thrombosis (2%). In the series reported none of these necessitated surgery or another invasive procedure.

Transient elevation of the LFT is common (93% of cases,), as up to 46% of patients develop a significant rise (AST twice baseline or higher), but levels generally return to normal within two weeks of the transplant⁴⁴. Pain is encountered during the procedure, mainly due to the intercostal access and the rise in the portal pressure. Pain is uncommon after the procedure⁴⁵.

Donor factors include age, preexisting islet damage trauma, unrecognized DM, amyloid, fat infiltration, prolonged ICU stay, hemodynamic stability and inotropic medication requirements. The quality of the organ procurement is important, including avoidance of warm ischemia and pancreatic capsular injury.

The cold ischemia time (between donor cross-clamping and the start of the isolation) should not exceed 8 hours with regular transport media. This includes the transport and the storage of the donor pancreas while immersed in the University of Wisconsin (UW) solution. A novel approach to organ preservation uses the two-layer preservation technique⁴⁶. This involves the use of two solutions—University of Wisconsin (UW) solution and perfluorodecalin. Perfluorodecalin is a perfluorocarbon which has the ability to store oxygen and slowly deliver it to the organ stored, thus preserving the cellular ATP content, which is important for cell viability in the context of organ storage. The two-layered technique enables longer cold ischemia times, with equivalent results when comparing 6-8 hours of storage in the UW solution with up to 24 hours of storage with the two-layered method⁴⁶. Factors that influence isolation of clinical grade islets include: Optimal enzyme batch³², temperature control during the process, reagent quality, and islet culture. Previously the present inventors have demonstrated that pancreatic duct preserved with M-Kyoto solution with ulinastatin⁴⁸ improved pancreatic ducal integrity which is essential for collagenase delivery. With this technique clinical grade islets were successfully isolated from non-heart-beating donors⁴⁸, therefore, transplantable islets can be obtained from heart-beating donors.

Clinical grade islet recovery is achieved in 18-35% of the pancreata used. The islet cell infusion delivers 40-85% of the normal cell mass, but engraftment is estimated at 25-50%⁴⁵. Therefore, a second islet cell infusion is necessary in most cases in order to achieve insulin independence. The total number of pancreatic islets transplanted influences the achievement of insulin-independence.

With the current isolation and preservation techniques infusion of a total of more than 9,000 islet-equivalents/kg is associated with a good graft outcome²⁷; this is typically achieved with the use of two donor pancreata. Recipient factors include anticoagulation and avoidance of cytokine activation and immunosuppression that avoids islet cell toxicity or insulin resistance.

The process of pancreatic islet isolation for transplant is performed in most centers in a specially designed facility in a clean environment using established protocols under the strict supervision of the FDA. The establishment of a new facility requires significant material investment followed by the appropriate validation process and necessitates skilled manpower ⁴⁷.

The focus of research in Islet Cell Transplants (ICT) is centered on the development of a safe and effective procedure that will eventually replace surgical pancreas transplantation together with an ideal immunosuppressive regimen that provides safe and effective prevention against rejection, while minimizing the side effects that negatively impact transplant recipient's quality of life.

Corticosteroids and high doses of calcineurin inhibitors as immunosuppressive agents have been associated with failure of the transplanted islets and return to insulin treatment. Using a regimen that provides adequate immunosuppression to prevent early and late rejection episodes, and minimizes steroid usage as well as high doses of calcineurin inhibitors as immunosuppressive agents is highly desirable.

This study is being conducted as a modification of the Edmonton protocol for ICT at our institution. Edmonton protocol is followed exception that: a) Etanercept and Anakinra may be administered during the early phase of the transplant to minimize the loss of islets due to inflammation which in turn will lead to improved islet engraftment; b) Thymoglobulin may be administered for induction instead of daclizumab; c) Sitaglipin (Januvia) may be used to enhance islet graft function. The use of Etanercept and Anakinra in this fashion is not described in the literature and to our knowledge is not currently applied in any islet cell protocol in this country. However, the expected side effect toxicity is low and potentially considerable immunologic advantage can be gained from this approach: namely, being able to decrease Rapamycin or Tacrolimus doses if there is toxicity from these two agents. This use of Etanercept and Anakinra is one of the main ways in which the current protocol is modified from Edmonton.

In addition, the present inventors developed a new islet isolation protocol originally developed for non-heart-beating donor pancreas in Japan. Especially, pancreatic ductal preservation at the time of pancreas procurement, trypsin inhibition during pancreas digestion and islet friendly purification solutions improve the quality and quantity of islets.

Procedures—Organ Procurement and Transport: The procurement of the pancreas for islet isolation is performed from a cadaveric donor as part of standard organ procurement according to the United Network for Organ Sharing (UNOS) guidelines in place nationwide. The organ procurement is performed by a qualified transplant surgery group in conjunction with a local Organ Procurement Organization. The surgeons and OPO must be familiar with harvesting and shipping pancreata for islet cell isolation. In addition, they must have the proper equipment and shipping materials for longer cold ischemia times.

The donor pancreas is shipped to the processing facility according to UNOS regulations for the standard donor pancreas. It is stored during the transport in University of Wisconsin (UW) solution alone or with oxygenated perfluorocarbon (PFC) solution or an appropriate shipping medium. Pancreatic duct is also preserved with M-Kyoto solution with ulinastatin³² or an appropriate preservation solution.

Every effort may be made to transplant the islet cells as soon as they are deemed ready by the laboratory team and the Medical Director in each and every instance. Study subjects will not be assigned different timelines for each of the steps of this study (procurement, isolation, recipient preparation, islet infusion). However, there are likely to be logistical delays at the donor operation, or in the laboratory work to separate the islets, or in the scheduling of the radiology suite, or in the preparation of the recipient. To prevent wastage of the cells, storage before isolation may be extended with the addition of perfluorocarbon to the University of Wisconsin solution, and storage after isolation but before transplantation may be extended with culture of the islets in an incubator. Because these timelines may vary somewhat from patient to patient, the differences in the time points between patients may be noted and correlated to success or failure to establish glycemic control. Likewise, the use of perfluorocarbon solution, and/or the use of culture of the islets may be correlated between patients.

Pancreatic Islet Isolation: Isolation of the islets from donor pancreata will occur in the Baylor Research Institute, Islet Cell Processing Laboratory (ICPL) using modified the “automated method” described by Ricordi, et al.³². The ICPL includes a Class 10,000 clean suite for processing islets, a QA/QC laboratory to perform product release testing and a freezer room to store samples and reagents. The ICPL has so far performed twenty nine islet isolations for validation. Furthermore, the laboratory has processed five islet products for transplants under a FDA approved protocol 11731A to test the safety and efficacy of remote site isolated islet products. The remote site validation protocol is simultaneously conducted in collaboration with the Diabetes Research Institute in Miami, Fla. Recently, ICPL performed 8 islet isolations with clinical grade pancreata and five isolated islets were successfully transplanted into four type 1 diabetic patients. More recently we performed three additional islet isolations for validation using collagenase enzyme from SERVA. Islet yield and the quality of all three isolations would have qualified for transplantation according to this protocol.

Human cadaveric donor pancreas may be received into the ICPL and islets may be isolated according to methods previously validated by the laboratory. All manipulations of the organ, islets and islet cell products are performed in Class 100 BioSafety cabinets which are contained in the class 10,000 clean suite.

These methods are as follows: Pancreas is acquired through an organ procurement organization (OPO) and shipped in Transport media. Preferably pancreatic duct is also preserved with M-Kyoto solution with Ulinastatin or an adequate preservation solution. The media will vary depending upon which OPO procures the organ. This varying media/transport may be carefully studied.

Validation Procedures—Release Testing Before Islet Infusion: Testing for each islet preparation final product includes islet cell counts, purity, viability, sterility, endotoxin and potency. The results of islet cell counts, purity, viability and endotoxin, are available prior to infusion, and must meet assay lot release criteria. The final results of the sterility and potency tests are not available until after infusion. If these results do not meet release criteria, corrective steps are taken as soon as the results are known. In addition, the product of islet isolation is tested prior to determining final disposition. If the interim tests do not pass release criteria, the cells will not be transplanted.

Islet Cell Infusion: Location. The islet cell infusion is performed in the Interventional Radiology Suite at Baylor University Medical Center or Baylor All Saints Medical Center by an interventional radiologist. The procedure takes place in a suite designed for invasive procedures using sterile technique with access to general anesthesia if necessary.

Preparation and Anesthesia: The patient is admitted and prepared for the procedure. Informed consents are obtained for the procedure.

The lower right lateral chest the upper right abdomen and the epigastric area are prepped sterile with iodine-based preparation. Local anesthesia with IV sedation usually suffices. Local anesthesia is performed using the anesthetic of choice as determined by the Interventional Radiologist, with intercostal nerve block of the area.

Cannulation of the portal vein: Guidance, for the portal vein cannulation is obtained with real-time ultrasonography using a 3.5 MHz probe.

Puncture site: The procedure is performed by percutaneous direct puncture of the liver. The right or the left branch of the portal vein can be chosen for cannulation and the puncture site is chosen accordingly by the interventional radiologist.

Technique: A 22G Chiba needle is used for access to the portal vein, following by the catheterization of the portal vein over a guide wire using the Seldinger technique. A 4-5Fr catheter is introduced in the portal vein. Needle and catheter size may change at the discretion of the interventional radiologist performing the procedure.

Portogram: A portal venogram is obtained through the catheter, with manual injection of low osmolar iodinated contrast, in order to evaluate anatomy and flow. Minimal contrast use is recommended.

Islet Cell Infusion—The Bag System: The islet cell infusion bag system is composed of a 600 mL infusion bag containing the islet suspension with a volume of 200 mL. The infusion of islet cells uses 1 or 2 bag systems. More than one bag is needed when the islet volume for infusion exceeds 5 mL. Each bag containing islets has 35 IU/kg heparin added. The maximum dose of heparin in the infusion is 70 IU/kg. If the infusion is terminated prematurely, the remainder of the heparin dose should be calculated to reach a total of 35 IU/kg and should be given into the portal vein followed by a normal saline flush.

The content of the bag is infused using gravitation only into the portal venous system of the recipient. The bag is then flushed with 50 mL of Transplant Media and the flush is infused from the bag into the portal system. The procedure is then repeated with the other bag or bags containing islets.

Completion of the Infusion: After the infusion is completed, the infusion catheter and the bag are rinsed with an additional transplant media, making sure that no islets are trapped in bag ports or 3-way stopcock. The portal venogram is not repeated after the infusion to avoid islet toxicity.

Portal Venous Pressure Assessment: The portal venous pressure is obtained by direct measurement inline via 3-way connector. Measures are read on a cardiovascular monitor after appropriate zeroing of the system.

Timing of Portal Vein Measurement: Portal vein (PV) pressures may be obtained before the procedure, halfway during each islet cell bag infusion and at the end of each wash of the bag with rinse solution. The final portal pressure is documented as well.

Management of Changes in Portal Venous Pressures: The portal venous pressure is expected to rise during the islet cell infusion. The following situations require adjustment of the treatment: Portal vein pressure above 20 mm Hg before the procedure is a contraindication for islet cell infusion.

If at any time during the infusion the PV pressure exceeds twice the baseline value but is less than 18 mm Hg, the infusion may be held for 10 minutes and the pressure may be measured again. If the pressure is below twice the baseline and less than 18 mm Hg the infusion may be resumed. If not, another measurement is made 10 minutes later.

If the PV pressure exceeds twice the baseline but is below 18 mm Hg the procedure may continue. If at any time the PV pressure exceeds 22 mm Hg, the infusion is held until the pressure falls below 18 mm Hg. If the PV pressure is above 22 mm Hg longer than 10 minutes, or above 18 mm HG more than 20 minutes, the procedure is terminated.

Removal of the Portal Vein Catheter: The portal vein catheter is removed and the introducer sheath is then withdrawn until the tip is in the parenchyma. A hemostatic agent of the

Radiologist's choice is placed in the tip of an iodine filled syringe and injected into external end of sheath. The hemostatic agent is further advanced to internal end of sheath using a stiffener/trocar/wire as chosen by the radiologist. The sheath is then withdrawn over the plug. The plug should be easily visualized within the liver parenchyma at this point. A second plug is placed if possible.

Recovery: Following the procedure the patient is observed in the Interventional Radiology recovery area for as long as necessary as determined by a Physician and then transferred to the Transplant Service for an overnight stay. Liver function tests and a Doppler ultrasonogram of the liver are obtained the day after the procedure.

Hospital stay: After recovery, the patient is admitted to the hospital on the Transplant Service for a 1-2 day observation. Length of stay may be determined by how the patient tolerates the initial dose of Thymoglobulin on Day 0. Patients will return to the hospital to receive subsequent dosing of Thymoglobulin on Day 2, 4 and 6 post-transplant. Criteria for discharge from hospital include: Laboratory test results which are not indicative of bleeding, including; but not exclusively; hemoglobin and hematocrit levels. LFT's within acceptable limits (less than twice upper limit of normal), and patent main, left and right PV with no significant bleed or collection per Doppler ultrasonogram performed the day after the islet cell infusion.

The present invention describes an effective newly designed cooling system to establish a new storage method for porcine islets. The inventors used the KFC cooling system which was originally developed by FUJIYA Co. for the preservation of plants and foods such as harvested orchids, fruits and shrimp. This cooling system was initially designed for the achieving ‘hibernation status’ for a long-term cooling preservation in the commercial agriculture field. The temperature settings of this cooling system can be easily changed and tightly controlled by an external sensing computer. The inventors have already reported that stepwise cooling by this system has some advantages for a whole rat liver preservation compared to conventional 4° C. preservation in UW solution (23). In this disclosure, the inventors demonstrate that KFC could effectively preserve porcine islets and maintained viability at least up to 72 hours for the first time.

The slow stepwise cooling by KFC could provide circumstances similar to hibernation. Indeed, some hibernating mammalians like squirrel or hamsters protect their metabolism (24,25) by equaling ATP synthesis rates and ATP utilization rates. This hibernation process could lead to stable ion gradients and regulate metabolic depression (26). Moreover, it has been shown that human myocardial cells can acquire ‘hibernation status’ (27). Therefore islet cells can acquire such hibernation status. Also the lowering the temperature reduces autolysis by cell-destructive enzymes like trypsin.

The inventors have demonstrated that the new KFC stepwise cooling system has advantages allowing porcine islets storage for up to 72 hours. Nevertheless, this KFC system is promising system to store fragile porcine islet cells.

It is contemplated that any embodiment discussed in this specification can be implemented with respect to any method, kit, reagent, or composition of the invention, and vice versa. Furthermore, compositions of the invention can be used to achieve methods of the invention.

It may be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it may be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. LaPorte R E, Matsushima M, Chang Y F: Prevalence and Incidence of Insulin-Dependent Diabetes. In: “Diabetes In America,” 2nd edition. National Diabetes Data Group, National Institutes of Health, National Institute of Diabetes and Digestive and Kidney Diseases, NIH Publication No. 95-1468, 1995, 37-47.

2. Ikemoto T, Noguchi H, Shimoda M, et al. Islet Cell Transplantation for the Treatment of Type 1 Diabetes in USA. J Hepato-biliary-Pancreatic Surg. 2009; 16: 118-123.

3. Ryan E A, Paty B W, Senior P A, et al. Five-year follow-up after clinical islet transplantation. Diabetes. 2005;54:2060-2069.

4. Matsumoto S, Okitsu T, Iwanaga Y, et al. Insulin independence after living-donor distal pancreatectomy and islet allotransplantation. Lancet.2005;365(9471):1642-1644.

5. Ichii H, Sakumaa Y, Pileggia A et al. Shipment of Human Islets for transplantation. Am J Transplant. 2007; 7: 1010-1020.

6. O'Neil J J, Stegemann J P, Nicholson D T, et al. The isolation and function of porcine islets from market weight pigs. Cell Transplant. 2001;10:235-46.

7. Calafiore R. Perspectives in pancreatic and islet cell transplantation for the therapy of IDDM. Diabetes Care. 1997;20:889-96.

8. Krickhahn M, Meyer T, Büler C, et al. Highly efficient isolation of porcine islets of Langerhans for xenotransplantation: numbers, purity, yield and in vitro function. Ann Transplant. 2001;6:48-54.

9. Bottino R, Balamurugan A N, Smetanka C, et al. Isolation outcome and functional characteristics of young and adult pig pancreatic islets for transplantation studies. Xenotransplantation. 2007;14:74.

10. Rood P P, Buhler L H, Bottino R, et al. Pig-to-nonhuman primate islet xenotransplantation: a review of current problems. Cell Transplant. 2006;15:89-104.

11. Matsumoto S, Kandaswamy R, Sutherland D E et al. Clinical application of the two-layer (University of Wisconsin solution/perfluorochemical plus O₂) method of pancreas preservation before transplantation. Transplantation. 2000;70:771-774.

12. Jacob S W. Studies in organ preservation by actual freezing and reduction of the freezing point. Cryobiology. 1964;1:176-80.

13. Obermaier R, Drognitz O, Benz S, et al. Pancreatic ischemia/reperfusion injury: impact of different preservation temperatures. Pancreas. 2008;37:328-32.

14. Frankel B J, Gylfe E, Hellman B, et al. Maintenance of insulin release from pancreatic islets stored in the cold for up to 5 weeks. J Clin Invest. 1976;57:47-52.

15. Korbutt G S, Pipeleers D G. Cold-preservation of pancreatic beta cells. Cell Transplant. 1994;3:291-7.

16. Matsumoto S, Lawrence O, Rigley T, et al. University of Wisconsin solution with trypsin inhibitor Pefabloc improves survival of viable human and primate impure islets during storage. Cell and Tissue Banking. 2001;2:15-21.

17. Kin T, Senior P, Gorman D O, et al. Risk factors for islet loss during culture prior to transplantation. Transplant International. 2008;21:1029-1035.

18. Ricordi C, Gray D W, Hering B J, et al. Islet isolation assessment in man and large animals. Acta Diabetol Lat. 1990;27:185.

19. Allen R D, Nankivell B J, Hawthorne W J, O'Connell P J, Chapman J R: Pancreas and islet transplantation: an unfinished journey. Transplant Proc 2001;33(7-8):3485-8.

20. Gruessner R W G, Sutherland D E R, Drangstveit M B, Bland B J, Gruessner A C: Pancreas transplants from living donors: short- and long-term outcome. Transplantation Proc 2001, 33 (1-2): 819-820.

21. Sutherland D E, Gruessner R W, Dunn D L, Matas, A J, Humar A, Kandaswamy R, Mauer S M, Kennedy W R, Goetz F C, Robertson R P, Gruessner A C, Najarian J S: Lessons learned from more than 1,000 pancreas transplants at a single institution. Ann Surg 2001, 233(4): 463-501.

22. Robertson R P, Sutherland D E, Lanz K J: Normoglycemia and preserved insulin secretory reserve in diabetic patients 10-18 years after pancreas transplantation. Diabetes 1999, 48(9): 1737-1740.

23. Fiorina P, La Rocca E, Astorri E, Lucignani G, Rossetti C, Fazio F, Giudici D, di Carlo V, Cristallo M, Pozza G, Secchi A: Reversal of left ventricular diastolic dysfunction after kidney-pancreas transplantation in type 1 diabetic uremic patients. Diabetes Care 2000, 23(12): 1804-1810.

24. Allen R D, Al-Harbi I S, Morris J G, Clouston P D, O'Connell P J, Chapman J R, Nankivell B J: Diabetic neuropathy after pancreas transplantation: determinants of recovery. Transplantation 1997, 63(6): 830-838.

25. Chow V C C, Pai R P, Chapman J R, O'Connell P J, Allen R D M, Mitchell P, Nankivell B J: Diabetic retinopathy after combined kidney-pancreas transplantation. Clinical Transplantation 1999, 13(4): 356-362.

26. Sollinger H W, Odorico J S, Knechtle S J, D'Alessandro A M, Kalayoglu M, Pirsch J D: Experience with 500 simultaneous pancreas-kidney transplants. Ann Surg 1998, 228(3):284-96.

27. Reddy K S, Stratta R J, Shokouh-Amiri M H, Alloway R, Egidi M F, Gaber A O: Surgical complications after pancreas transplantation with portal-enteric drainage. J Am Coll Surg 1999;189(3): 305-313.

28. Gruessner A C, Sutherland D E: Pancreas transplant outcomes for United States (US) cases reported to the United Network for Organ Sharing (UNOS) and non-US cases reported to the International Pancreas Transplant Registry (IPTR) as of October, 2000 . Clinical Transplants 2000, (1): 45-72.

29. 2001 Annual Report of the U.S. Organ Procurement and Transplantation Network and the Scientific Registry for Transplant Recipients: Transplant Data 1991-2000. Department of Health and Human Services, Health Resources and Services Administration, Office of Special Programs, Division of Transplantation, Rockville, Md.; United Network for Organ Sharing, Richmond, Va.; University Renal Research and Education Association, Ann Arbor, Mich.

30. Linetsky E, Bottino R, Lehmann R, Alejandro R, Inverardi L, Ricordi C: Improved human islet isolation using a new enzyme blend, liberase. Diabetes 1997,46(7):1120-1123.

31. Lakey J R T, Warnock G L, Shapiro A M J, Korbutt G S, Ao Z, Kneteman N M, Rajotte R V: Intraductal collagenase delivery into the human pancreas using syringe loading or controlled perfusion. Cell Transplant 1999, 8(3):285-292.

32. Ricordi C, Lacy P E, Scharp D W: Automated islet isolation from human pancreas. Diabetes 1989, 38 (Suppl. 1): 140-142.

33. Shapiro A M J, Lakey J R T, Ryan E A, Korbutt G S, Toth E L, Warnock G L, Kneteman N N, Rajotte R V: Islet transplantation in seven patients with Type 1 diabetes mellitus using a glucocorticoid-free immunosuppressive regimen. N Engl J Med 343:230-238, 2000.

34. Rutzky L P, Bilinski S, Kloc M, Phan T, Zhang H, Katz S M, Stepkowski S M: Microgravity culture condition reduces immunogenicity and improves function of pancreatic islets. Transplantation 2002 Jul. 15, 74(1): 13-21.

35. Gaber A O, Fraga D W, Callicutt C S, Gerling I C, Sabek O M, Kotb M Y: Improved in vivo pancreatic islet function after prolonged in vitro islet culture. Transplantation 2001, 72(11): 1730-1736.

36. Matarazzo M, Giardina M G, Guardasole V, Davalli A M, Horton E S, Weir G C, Sacca L, Napoli R: Islet transplantation under the kidney capsule corrects the defects in glycogen metabolism in both liver and muscle of streptozocin-diabetic rats. Cell Transplant 2002, 11(2): 103-112.

37. Hirshberg B, Montgomery S, Wysoki M G, Xu H, Tadaki D, Lee J, Hines K, Gaglia J, Patterson N, Leconte J, Hale D, Chang R, Kirk A D, Harlan D M: Pancreatic islet transplantation using the nonhuman primate (rhesus) model predicts that the portal vein is superior to the celiac artery as the islet infusion site. Diabetes 2002, 51(7): 2135-2140.

38. Levy M M, Ketchum R J, Tomaszewski J E, Naji A, Barker C F, Brayman K L: Intrathymic islet transplantation in the canine: I. Histological and functional evidence of autologous intrathymic islet engraftment and survival in pancreatectomized recipients. Transplantation 2002, 73(6): 842-852.

39. Drachenberg C B, Klassen D K, Weir M R, Wiland A, Fink J C, Bartlett S T, Cangro C B, Blahut S, Papadimitriou J C: Islet cell damage associated with tacrolimus and cyclosporine: morphological features in pancreas allograft biopsies and clinical correlation. Transplantation 1999, 68(3): 396-402.

40. Ricordi C, Strom, T B: Clinical islet transplantation: Advances and immunological challenges. Nat Rev Immunol. 2004 April;4(4):259-68.

41. Froud T, Ricordi C, Baidal D A, Hafiz M H, Ponte G, Cure P, Pileggi A, Poggioli R, Ichii H, Khan A, Ferreiraa J V, Pugliese A, Esquenazi V V, Kenyon N S, Alejandro R. Islet transplantation in type 1 diabetes mellitus using cultured islets and steriod-free immunosuppression: Miami experience. Am J Transplantation 2005 5(8):2037-2046

42. Hering B J, Kandaswamy R, Ansite J D, Eckman P M, Nakano M, Sawada T, Matsumoto I, Ihm S-H, Zhang H-J, Parkey J, Hunter D W, Sutherland D E R. Single-donor, marginal-dose islet transplantation in patients with type 1 diabetes JAMA 2005 293(7):830-835

43. Markmann J F, Rosen M, Sigelman E S, Soulen M C, Deng S, Barker C F, Naji A. Magnetic resonance-defined periportal steatosis following intraportal islet transplantation: a functional footprint of islet graft survival? Diabetes 2003 52(7):1591-1594

44. Ryan E A, Lakey J R, Rajotte, R V, Korbutt G S, Kin T, Imes S, Rabinovitch A, Elliott J F, Bigam D: Kneteman N M, Warnock G L, Larsen I, Shapiro A M: Clinical outcomes and insulin secretion after islet transplantation with the Edmonton protocol. Diabetes 2001, 50(4): 710-719.

45. Owen, R J T, Ryan, E A, O'Kelly, K, Lakey, J R T, McCarthy, M C, Paty, B W, Bigam, D L, Kneteman, N M, Korbutt, G S, Rajotte, R V, Shapiro, A M J: Percutaneous transhepatic pancreatic islet cell transplantation in type 1 diabets mellitus: Radiologic aspects. Radiology 2003, 229: 165-170.

46. Matsumoto S, Qualley S A, Goel S, Hagman D K, Sweet I R, Poitout V, Strong D M, Robertson R P, Reems J A: Effect of the two-layer (University of Wisconsin solution-perfluorochemical plus O2) method of pancreas preservation on human islet isolation, as assessed by the Edmonton Isolation Protocol. Transplantation 2002, 74(10): 1414-1419

47. Rastellini C, Braun M, Cicalese L, Benedetti E: Construction of an optimal facility for clinical pancreatic islet isolation. Transplant Proc 2001, 33(7-8): 3524.

48. Matsumoto S, Okitsu T, Iwanaga Y, Noguchi H, Nagata H, Yonekawa Y, Yamada Y, Fukuda K, Shibata T, Kasai Y, Maekawa T, Wada H, Nakamura T, Tanaka K. Successful islet transplantation from nonheartbeating donor pancreata using modified Ricordi islet isolation method. Transplantation 2006 82(4):460-5.

49. Ryan E A, Shandro T, Green K, Paty B W, Senior P A, Bigam D, Shapiro A M, Vantyghem M C. Assessment of the severity of hypoglycemia and glycemic lability in type 1 diabetic subjects undergoing islet transplantation. Diabetes. 2004 April;53(4):955-62.

50. Levy M F, Jennings L, Abouljoud M S, Mulligan D C, Goldstein R M, Husberg B S, Gonwa T A, Klintmalm G B. Quality of life improvements at one, two, and five years after liver transplantation Transplantation 1995 59(4):515-518.

What is claimed is:

1. An apparatus for storing organs, tissues, or cells in which the organs, tissues, or cells are suspended in a solution for maintaining viability and the organs, tissues, or cells are cooled in a refrigeration unit for the entire duration of storage in which the average temperature in the apparatus does not vary by more than 2 degrees centigrade from the set temperature.

2. The apparatus of claim 1, wherein the apparatus cools the organ or tissue from body temperature to about 4° C. within 18 minutes.

3. The apparatus of claim 1, wherein the apparatus further comprises one or more portals for a preservation gas selected from CO₂, N₂ or O₂.

4. The apparatus of claim 1, wherein the apparatus comprises one or more probes that determine the organ or cell temperature.

5. The apparatus of claim 1, wherein the apparatus vary the temperature in the apparatus no more that 1 degree centigrade from the set temperature.

6. The apparatus of claim 1, wherein the apparatus vary the temperature in the apparatus no more that 0.5 degrees centigrade from the set temperature.

7. The apparatus of claim 1, wherein the apparatus has a set temperature of greater than 0, 1, 2, 3, 4, 5 or 6 degrees centigrade.

8. The apparatus of claim 1, wherein the organ or tissue comprises at least a portion of a liver, a lung, a cornea, a muscle, a heart, a pancreas, pancreatic islets, a kidney, a breast, an eye, an ear, a bone or a bone marrow. In another aspect, the organ is treated during storage with one or more active agents that will enhance organ transplant.

9. The apparatus of claim 1, wherein a viability of the stored organs or tissues is at least 80%.

10. The apparatus of claim 1, wherein the viability of the stored organ or tissue is 100%, 95%, 90%, 85%, 80%, 75%, 70%, 60%, 50%, 40% and 30%.

11. The apparatus of claim 1, wherein the organ or tissue is treated during storage with one or more active agents selected from antibodies, enzymes, steroids, antibiotics, proteases, nucleases, vectors, nucleic acids, proteins, peptides, lipids, carbohydrates, salts, minerals, vitamins, buffers, gases, electrical impulses, mechanical stress (extension and/or compression), radiation or toxins.

12. A method of preserving an organ or tissue comprising:
obtaining an organ or tissue for transplant;
placing the organ or tissue in an preservation solution;
cooling the organ or tissue to a pre-selected temperature; and
maintaining the organ or tissue at the pre-selected temperature for the duration of storage at a temperate that does not vary more that 2 degrees centigrade from the pre-selected temperatures.

13. The method of claim 12, wherein the apparatus cools the organ or tissue from body temperature to about 4° C. within 18 minutes.

14. The method of claim 12, wherein the apparatus further comprises one or more portals for a preservation gas selected from CO₂, N₂ or O₂.

15. The method of claim 12, wherein the apparatus comprises one or more probes that determine the organ or cell temperature.

16. The method of claim 12, wherein the apparatus vary the temperature in the apparatus no more that 1 degree from the set temperature.

17. The method of claim 12, wherein the apparatus vary the temperature in the apparatus no more that 0.5 degrees centigrade from the set temperature.

18. The method of claim 12, wherein the apparatus has a set temperature of greater than 0, 1, 2, 3, 4, 5 or 6 degrees centigrade.

19. The method of claim 12, wherein the organ or tissue comprises at least a portion of a liver, a lung, a cornea, a muscle, a heart, a pancreas, pancreatic islets, a kidney, a breast, an eye, an ear, a bone or a bone marrow.

20. The method of claim 12, wherein the preserved organ or tissue has a viability of at least 80%.

21. The method of claim 12, wherein the viability of the preserved organ or tissue is 100%, 95%, 90%, 85%, 80%, 75%, 70%, 60%, 50%, 40% and 30%.

22. The method of claim 12, wherein the organ or tissue is treated during storage with one or more active agents that will enhance organ transplant. In another aspect, the organ is treated during storage with one or more active agents selected from antibodies, enzymes, steroids, antibiotics, proteases, nucleases, vectors, nucleic acids, proteins, peptides, lipids, carbohydrates, salts, minerals, vitamins, buffers, gases, electrical impulses, mechanical stress (extension and/or compression), radiation or toxins.

23. A method of preparing a transplantable islet preparation, the method comprising the steps of:
harvesting a pancreas or a pancreatic tissue from a donor;
injecting one or more pancreatic ducts with ET-Kyoto solution or equivalent thereto;
isolating pancreatic β-islet cells;
cooling the pancreas or pancreatic tissue to a pre-selected temperature; and
maintaining the pancreas or pancreatic tissue at the pre-selected temperature in an apparatus for the duration of storage at a temperate that does not vary more that 2 degrees centigrade from the pre-selected temperatures.

24. The method of claim 23, wherein the apparatus varies the temperature in the apparatus no more that 1 degree centigrade from the set temperature.

25. The method of claim 23, wherein the apparatus varies the temperature in the apparatus no more that 0.5 degrees centigrade from the set temperature.

26. The method of claim 23, wherein the apparatus has a set temperature of greater than 0, 1, 2, 3, 4, 5 or 6 degrees centigrade.

27. The method of claim 23, wherein the step of isolating the pancreatic β-islet cells is performed with a collagenase.

28. The method of claim 23, wherein the isolated pancreatic β-islet cells have a recovery rate of at least 35%.

29. The method of claim 23, wherein the recovery rate of the isolated pancreatic β-islet cells is 100%, 95%, 90%, 85%, 80%, 75%, 70%, 60%, 50%, 40%, 35%, 30%, 25%, and 20%.

30. The method of claim 23, wherein the isolated pancreatic β-islet cells have a purity of at least 70%.

31. The method of claim 23, wherein the purity of the isolated pancreatic β-islet cells is 100%, 95%, 90%, 85%, 80%, 75%, 70%, 60%, 50%, 40%, 35%, 30%.

32. The method of claim 23, wherein the isolated pancreatic β-islet cells have a viability of at least 80%.

33. The method of claim 23, wherein the viability of the isolated pancreatic β-islet cells is 100%, 95%, 90%, 85%, 80%, 75%, 70%, 60%, 50%, 40% and 30%.

34. The method of claim 23, wherein the human interleukin-1 antagonist is selected from: one or more modifiers of interleukin-1 beta (IL-1β) gene transcription; one or more modifiers of IL-1β gene translation; one or more siRNAs that target the expression of IL-1β; one or more IL-1β receptors blockers; one or more interleukin-1 receptor antagonist proteins; one or more interleukin-1 receptor antagonist peptides; one or more active agents that modify the release of IL-1β; one or more antibodies that neutralize IL-1β; one or more antibodies that blocks an IL-1β receptor; one or more recombinant, naturally occurring IL-1 receptor antagonists; one or more anion transport inhibitors, lipoxins and alpha-tocopherol that inhibit the release of IL-1β; one or more opioids that inhibits a proteolytic enzyme that converts the inactive IL-1β precursor to its mature, active form; one or more antibodies that neutralizes the biological function of IL-1β, mixtures and combinations thereof

35. The method of claim 23, further comprising providing the patient with a Tumor Necrosis Factor antagonist, selected from inhibitors of gene transcription, inactivated Tumor Necrosis Factors, Tumor Necrosis Factor Receptor blockers and soluble Tumor Necrosis Factor Receptor.

36. A method of storing a transplantable organ or tissue, the method comprising the steps of:
harvesting a transplantable organ or tissue;
placing the organ or tissue in a preservation solution;
cooling the organ or tissue to a pre-selected temperature; and
maintaining the organ or tissue at the pre-selected temperature for the duration of storage at a temperate that does not vary more that 1 degree centigrade from the pre-selected temperatures.

37. The method of claim 36, wherein the transplantable organ or tissue has a viability of at least 80%.
